(19) United States
(12) Patent Application Publication
(10) Pub. No.: US 2025/0040846 A1
(43) Pub. Date: Feb. 6, 2025

(54) Keyboard-Based Health Assessment and Feedback

(71) Applicant: KeyWise, Inc., Chicago, IL (US)

(72) Inventors: Steve Curd, Chicago, IL (US); Raeanne C. Moore, Chicago, IL (US); Olusola Ajilore, Chicago, IL (US); Mark Heinemeyer, Chicago, IL (US)

(21) Appl. No.: 18/792,900

(22) Filed: Aug. 2, 2024

Related U.S. Application Data
(60) Provisional application No. 63/530,661, filed on Aug. 3, 2023.

(51) Int. Cl. A61B 5/16; G06F 3/04817; G06F 3/04842; G06F 3/04886; G06F 11/34; G06T 11/60

(52) U.S. Cl. CPC A61B 5/16; G06F 3/04817; G06F 3/04842; G06F 3/04886; G06F 11/3438; G06T 11/60

(57) ABSTRACT

Examples described herein relate to cognitive or other health-related feedback displayed on a digital keyboard. A digital keyboard may collect dynamic keystroke data and use this data to determine one or more measures indicative of health. This measure (or measures) may be displayed on the digital keyboard concurrently with the keys so as to provide health-related feedback while the digital keyboard is in use (e.g., while the user is typing) and without the user having to leave their current app. The digital keyboard may further be configured to display additional health information in lieu of the keys when a particular control (e.g., an icon showing the measure) is selected.

### CROSS-REFERENCE TO RELATED APPLICATION(S)

The present application claims the benefit of priority to U.S. Provisional Patent Application No. 63/530,661, filed Aug. 3, 2023, which is incorporated by reference herein in its entirety.

### BACKGROUND

Brain health typically is assessed through clinical evaluations, diagnostic interviews, mood ratings, and other assessments that are conducted intermittently and in a controlled environment. These assessments frequently depend on a patient's self-reported symptoms and/or symptoms reported by a relevant third party (e.g., family members, caretakers, etc.), resulting in the reported symptoms being subject to recall biases, thereby making diagnoses less reliable. In addition, the reported symptoms may also often be representative of a particular period in time or sporadic, irregular periods in time and thus might not accurately illustrate the patient's symptoms and condition as a whole.

### SUMMARY

Example technologies described herein relate to cognitive feedback displayed on a digital keyboard. A digital behavior keyboard may collect dynamic keystroke data and use this data to determine one or more measures indicative of cognitive health. An example of such a measure is a composite cognitive measure based on two or more cognitive metrics. This measure (or measures) may be displayed on the digital keyboard concurrently with the keys so as to provide direct cognitive feedback while the digital keyboard is in use. The digital keyboard may further be configured to display additional information in lieu of the keys when particular controls (e.g., an icon showing the measure) are selected.

Accordingly, a first example embodiment may involve initiating, on a computing device, an application that adds a digital behavior keyboard to the computing device, wherein, when enabled, the digital behavior keyboard replaces a digital application keyboard on the computing device, and wherein the digital behavior keyboard comprises keys that are selectable via a touchscreen of the computing device to provide input to other applications; while the digital behavior keyboard is enabled, measuring, via the digital behavior keyboard, user interaction features with the digital behavior keyboard, wherein the user interaction features comprise dynamic keystroke data representative of user keyboard usage patterns made while providing keystroke input via the keys to one or more of the other applications; based on the user interaction features including the dynamic keystroke data, determining, via the digital behavior keyboard, a composite cognitive measure for a user; and displaying, by the computing device on the touchscreen, the composite cognitive measure as an icon directly on a portion of the digital behavior keyboard concurrently with the keys.

In a second example embodiment, an article of manufacture may include a non-transitory computer-readable medium, having stored thereon program instructions that, upon execution by a computing device, cause the computing device to perform operations in accordance with the first example embodiment.

In a third example embodiment, a computing device may include at least one processor, as well as memory and program instructions. The program instructions may be stored in the memory, and upon execution by at least one processor, cause the computing system to perform operations in accordance with the first and/or second example embodiment.

In a fourth example embodiment, a system may include various means for carrying out each of the operations of the first, second, and/or third example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 depicts a computing device, in accordance with example embodiments.

FIG. 2A depicts a user interface of a computing device, in accordance with example embodiments.

FIG. 2B depicts data collected by a computing device, in accordance with example embodiments.

FIG. 3A depicts a user interface of a computing device, in accordance with example embodiments.

FIG. 3B depicts data collected by a computing device, in accordance with example embodiments.

FIG. 4 depicts a user baseline model, in accordance with example embodiments.

FIG. 5 depicts a user baseline model, in accordance with example embodiments.

FIG. 6 depicts a user baseline model, in accordance with example embodiments.

FIGS. 7A, 7B, and 7C depict example user interfaces, in accordance with example embodiments.

FIGS. 8A, 8B, and 8C depict example user interfaces, in accordance with example embodiments.

FIG. 9 depicts an example user interface of an application that adds a digital behavior keyboard, in accordance with example embodiments.

FIGS. 10A, 10B, 10C, 10D, and 10E depict an example digital behavior keyboard, in accordance with example embodiments.

FIG. 11 is a flow chart of a method, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Overview

Treatment of neuropsychiatric disorders may have been hampered by the lack of objective tests of brain function relevant to such disorders. Current methods of assessing the course of these neuropsychiatric disorders and assessing the course of treatment of these neuropsychiatric disorders may be limited by biases including recency bias and recall bias. Further, these assessments may be done asynchronously, which may miss vital temporal features of symptomatic changes. These limitations may contribute to unsatisfactory clinical outcomes for patients with these disorders, as well as hampering the development of treatments for these disorders.

Described herein are technologies that take advantage of recent developments, such as the proliferation of mobile devices and the advancements in machine learning, to address these limitations. An application on a mobile device may collect digital behaviorome data on user activities using the mobile device, and the application may assess the collected digital behaviorome data for physical, emotional, and/or cognitive user functions using machine learning algorithms and statistical techniques.

In some examples, a computing device may receive digital behaviorome data collected using sensors associated with the computing device. For example, the sensors may include a touchscreen, a keyboard, one or more gyroscopes, one or more accelerometers, other sensors, and/or a combination thereof. Digital behaviorome data collected using these sensors may include a plurality of user interaction features, which may include keystroke dynamic data representative of user keyboard usage patterns. For example, the plurality of user interaction features may include individual keystrokes, transitions between individual keystrokes, pauses between keystrokes, number of pauses between keystrokes, backspace usage features, input mistakes features, input time features, typing rhythm features, accuracy features, and so on. These individual keystrokes, transitions between individual keystrokes, and pauses between keystrokes may be collected as keystroke dynamic data.

The computing device may passively collect this digital behaviorome data as the user is using the computing device during normal operations. For example, a user may be typing a message to post on social media using the computing device. The computing device may collect keystroke dynamic data and other keystroke information using the touchscreen on the computing device as the user is typing out the message to post on social media. In some examples, the computing device may also collect data using an accelerometer and/or a gyroscope on the computing device to determine whether the user set the computing device down (e.g., on a table or other surface), the angle at which the computing device is tilted while the user is typing out the note, and so on.

In some instances, the computing device may collect the digital behaviorome data via a digital behavior keyboard. A digital behavior keyboard may be similar to other digital on-screen keyboards, referred to herein as digital application keyboards, in that these keyboards provide a user interface with selectable buttons or other controls to input keystrokes. Such controls are referred to herein as “keys.” In addition to these keyboard functions, the digital behavior keyboard collects the digital behaviorome data as the user is interacting with the user interface (e.g., typing using the keys). In this manner, the digital behavior keyboard can be considered a specialized digital application keyboard.

Based on the digital behaviorome data, the computing device may determine one or more user baseline models. Each of the user baseline models may include statistical relationships between at least two of the user interaction features and correspond to one or more physical, emotional, and/or cognitive user characteristics. In some examples, a user baseline model may include an expected distribution of inter-key delay and the frequency of the various keystroke transitions for normal and/or regular user cognitive function. In further examples, a user baseline model may include an expected clustering of points representing inattentiveness, where the cluster of points may be represented by a range of values in a high dimensional space representing the relationship between inter-key delay and frequency. And in some examples, a user baseline model may include an expected clustering of points representing the expected distribution of points as a disease progresses, where the model includes relationships between frequency, time of day, and inter-key delay. Further, in some examples, a user baseline model may be a probabilistic graphical model (e.g., a Hidden Markov Model (HMM)) that may predict a cognitive process based on the digital behaviorome data, and the cognitive process may be associated with a particular model that represents relationships between a plurality of user interaction features.

The computing device may receive additional digital behaviorome data that includes a plurality of additional user interaction features. The additional digital behaviorome data may be compared against the user baseline models described above to determine whether the user displays normal and/or abnormal physical, emotional, and/or cognitive user characteristics.

Specifically, the computing device may select a particular user baseline model from the one or more user baseline models based on the particular user baseline model including statistical relationships between the plurality of additional user interaction features with which the digital behaviorome data is associated. For example, if the additional digital behaviorome data includes inter-key delay and the frequency of each inter-key delay, the computing device may then select a user baseline model that includes relationships between inter-key delay and the frequency of each inter-key delay, e.g., a user baseline model including an expected clustering of points representing inattentiveness as described by certain relationships between inter-key delay and the frequency of each inter-key delay.

The computing device may then determine a statistical value based on a comparison of the additional user interaction features relative to the particular user baseline model. For example, the computing device may determine the likelihood that the additional inter-key delay and the additional frequency of the various keystroke transitions of the additional digital behaviorome data falls within the cluster that represents inattentiveness. For instance, if the additional inter-key delay and the additional frequency of the various keystroke transitions falls within the cluster representing inattentiveness, then the statistical value may be one. Whereas, if the additional inter-key delay and the additional frequency of the various keystroke transitions falls outside the cluster representing the inattentiveness, then the statistical value may be zero, or if the additional inter-key delay and the additional frequency of the various keystroke transitions falls on the border of the cluster representing the inattentiveness, then the statistical value may be between zero and one, depending on how close the additional inter-key delay and the additional frequency of the various keystroke transitions falls in or out of the cluster.

The computing device may determine whether the determined statistical value is outside a predefined range to determine whether the physical, emotional, or cognitive user characteristic for the user is within an expected range. For example, the determined statistical value may be a number, indicating that the additional inter-key delay and the additional frequency of the various keystroke transitions falls outside of the cluster. In some examples, the predefined range for the determined statistical value may be from a first number to a second number. Thus, if the number of the determined statistical value is not within the range of the first number to the second number, then based on this determined statistical value being outside the predefined range, the computing device may determine that the physical, emotional, and/or cognitive function of the user is within the expected range (e.g., the user is not having inattentiveness).

The computing device may then display this information to the user. For example, the computing device may display on its user interface that the user is likely not being inattentive or having slowed thinking. In some examples, the computing device may also display other physical, emotional, and/or cognitive functions of the user, the trends of these physical, emotional, and/or cognitive functions (e.g., having more inattentiveness on Monday than Tuesday), recommendations to improve physical, emotional, and/or cognitive functions (e.g., more sleep, mindful breathing), a combination thereof, and/or other information/recommendations to the user.

In some examples, the digital behavior keyboard may provide a graphical representation of the information. For instance, the digital behavior keyboard may provide a graphical representation of a composite cognitive measure. The composite cognitive measure may be a numerical value representing a composite of two or more determined statistical values indicating a user's cognitive, emotional, or physical functions, which are based on user keyboard usage patterns associated with processing speed and executive function. Certain examples of the composite cognitive measure are referred to herein as a Clarity Score.

Within examples, the composite cognitive measure is displayed on the keyboard concurrently with the keys. Such an arrangement facilitates real-time visual feedback to the user of their physical, emotional, and/or cognitive function while the digital behavior keyboard is in use. Since a digital keyboard may be of a fixed or limited size, summarizing the user's physical, emotional, and/or cognitive function with a single measure (e.g. a composite cognitive measure) facilities providing such feedback without interfering with the user's use of the keyboard as an input interface.

In some examples, the composite cognitive measure is displayed on a selectable control (e.g., an icon) concurrently with the keys. Further, in some examples, selection of this icon may display additional information, such as the afore-mentioned trends of the physical, emotional, and/or cognitive function of the user when the icon or other graphical representation is selected. Within examples, this additional information may replace other aspects of the keyboard (e.g., the keys) to as to provide space to show such information within the keyboard user interface, which may be of a fixed or otherwise limited size to allow concurrent display of another application on-screen. The user may then select the same control (or another suitable control on the digital behavior keyboard) to revert the keyboard user interface back to the keys.

### II. Example Computing Devices and Cloud-Based Computing Environments

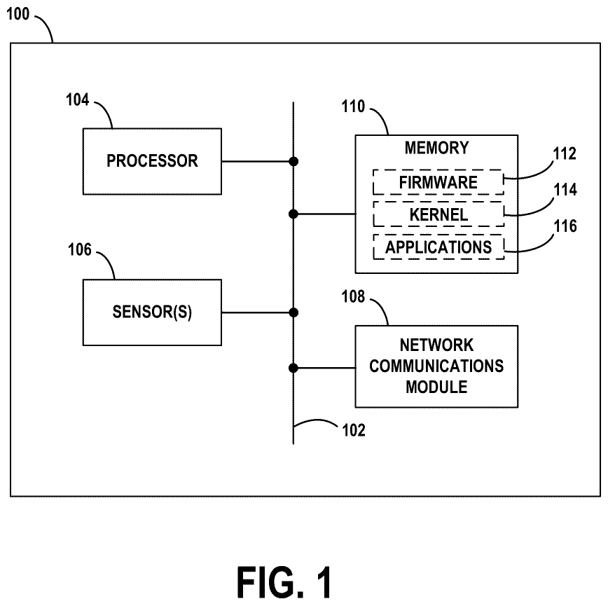

FIG. 1 is a simplified block diagram depicting an example computing device 100, illustrating some of the components that may be included in a computing device arranged to operate in accordance with the embodiments herein. The computing device 100 may be a user device (e.g., a device actively operated by a user), such as a mobile device (such as a tablet computer, a smartphone, or a wearable computing devices) or a (relatively-more) stationary device (such as a laptop or desktop computer).

In this example, the computing device 100 includes a processor 104, one or more sensor(s) 106, a network communications module 108, and memory 110, all of which may be connected by system bus 102 or a similar mechanism. In some examples, the computing device 100 may include other components and/or peripheral devices (e.g., keyboards, sensors, detachable storage, printers, etc.). Additionally or alternatively, components of the computing device 100 may have the ability to be decoupled. For example, the sensor(s) 106 may include a detachable keyboard that connects to computing device 100.

In some examples, the processor 104 may be one or more of any type of computer processing element, such as a central processing unit (CPU), a co-processor (e.g., a graphics processing unit), a network processor, and/or a form of integrated circuit or controller that performs processor operations. The processor 104 may be one or more single-core processors and/or one or more multi-core processors with multiple independent processing units. In some examples, the processor 104 may include multiple types of processors. The processor 104 may also include register memory for temporarily storing instructions being executed and related data, as well as cache memory for temporarily storing recently-used instructions and data.

The sensor(s) 106 may include one or more of any type of sensor used in operations of computing device 100. For example, the sensor(s) 106 may include gyroscopes, accelerometers, cameras, touchscreens, tactile buttons, keyboards, and so on. The sensor(s) 106 may be integrated onto the computing device 100 (e.g., soldered onto a printed circuit board of the computing device 100) or be temporarily attached onto the computing device 100 (e.g., a removable keyboard or camera connected to computing device 100 via a USB peripheral). The computing device 100 may collect data from sensor(s) 106 and store them in the memory 110.

The memory 110 may be any form of computer-usable memory, including but not limited to random access memory (RAM), read-only memory (ROM), and non-volatile memory (e.g., flash memory, hard disk drives, solid state drives, compact discs (CDs), digital video disks (DVDs), and/or tape storage). Thus, the memory 110 may represent both temporary storage units, as well as long-term storage.

The memory 110 may store program instructions and/or data on which program instructions may operate. By way of example, the memory 110 may store these program instructions on a non-transitory computer-readable medium, such that the instructions are executable by processor 104 to carry out any of the methods, processes, or operations disclosed in this specification or the accompanying drawings.

As shown in FIG. 1, the memory 110 may include firmware 112, a kernel 114, and/or applications 116. The firmware 112 is program code used to boot or otherwise initiate some or all of computing device 100. The kernel 114 is an operating system, including modules for memory management, scheduling, and management of processes, input/output, and communication. The kernel 114 may also include device drivers that allow the operating system to communicate with the hardware modules (e.g., memory units, networking interfaces, ports, and buses) of computing device 100. The applications 116 include one or more user-space software programs, such as web browsers or email clients, as well as any software libraries used by these programs. The memory 110 may also store data used by these and other programs and applications.

The network communications module 108 facilitates wireless communications (e.g., IEEE 802.11 (Wi-Fi), BLUETOOTH®, global positioning system (GPS), a wide-area wireless interface, or so on) and/or wired communications (e.g., Ethernet, Synchronous Optical Networking, digital subscriber line, and so on). In some examples, the network communications module 108 includes one or more network communications modules and support one or more wireless and/or wired communications methods. For example, the network communications module 108 may include a module that supports Wi-Fi® and a module (separate or integrated) that supports BLUETOOTH®. As another example, the network communications module 108 may include a module that supports Wi-Fi and a module (separate or integrated) that supports Ethernet.

### III. Example Data Collection and Analysis

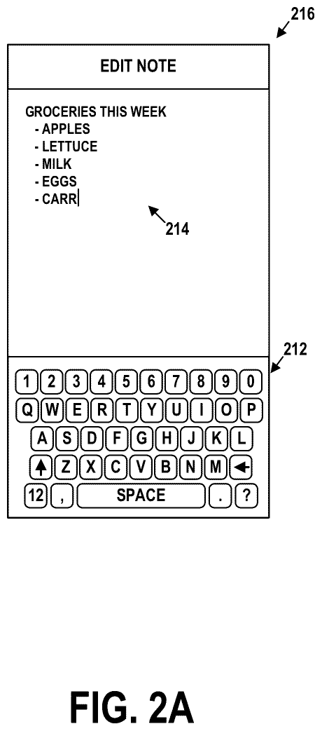

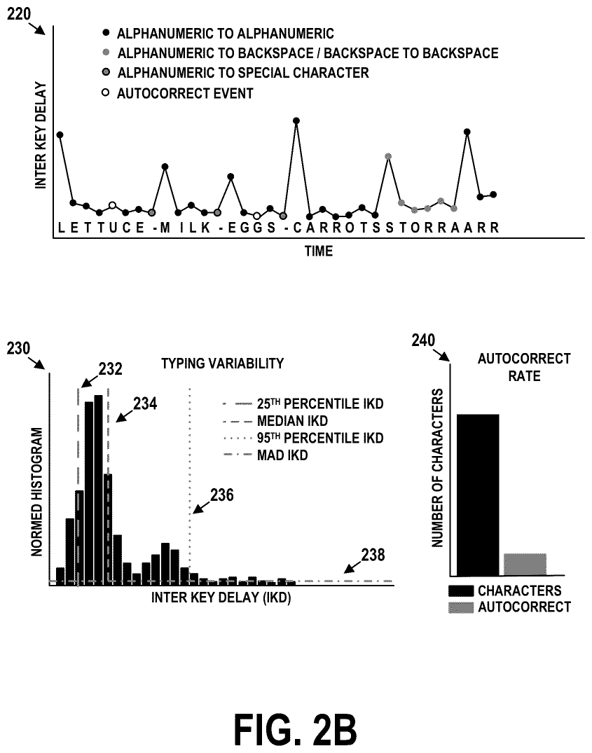

FIG. 2A depicts a touch-sensitive display 216 of the computing device 100, in accordance with example embodiments. The computing device 100 may include one or more sensors (e.g., gyroscope(s), accelerometer(s), touchscreen(s), push button(s), and so on) that may be used to collect digital behaviorome data. While a user is interacting with the computing device 100, the computing device 100 passively collects digital behaviorome data using the sensors associated with computing device 100. The digital behavorome data includes a plurality of user interaction features. For example, the user interaction features may include measurements collected from gyroscopes, accelerometers, and so on. The user interaction features may also include keystroke dynamic data representative of user keyboard usage patterns.

The users of devices with touch-sensitive screens, including but not limited to cellular phones, computer displays and tablet computers, typically interact with the device using various categories of motion, including the following exemplary interactions:

Taps: by tapping an image or graphic on the device's screen, the user can communicate some form of selection, such as launching an application (App), ticking a checkbox, operating a button, or choosing an item from a list of multiple items.
Gestures: by touching the device screen then sliding one's finger (or multiple fingers) in a particular direction, a user can communicate an action depending upon the direction of the “swipe”. For example, swiping left or right can signal moving to the next or previous screen of information. Swiping up or down can signal scrolling through continuous content that spans multiple screen views. In some cases, swiping may enable the user to delete an item, or indicate a positive or negative reaction to an item.
Physical motion: by shaking the device, or moving it physically through space, the user may initiate specific actions, such as erase previously entered content, or navigate the device to a different app or state.

To provide richer content entry than simple navigation or selection motion allows, the use of a virtual keyboard is often combined with taps and gestures to enable the user to input textual information. In this embodiment, an image of a familiar keyboard is displayed on the screen of the device, and the user taps images of the individual characters on the screen to select and enter characters, much like “typing” on a traditional keyboard commonly used with laptop and desktop computers.

For example, the computing device 100 may include a touchscreen that, at times, may display a keyboard application 212, as well as other applications that are not shown. A user may use the keyboard application 212 to type out text, such as a note 214. While the user is using the keyboard application 212, the keyboard application 212 collects and analyzes keystroke dynamic data representative of user keyboard usage patterns. Specifically, the keystroke dynamic data may include data collected directly from a keyboard application (e.g., the keyboard application 212) and/or data collected from a keyboard application that has been analyzed. For example, the keystroke dynamic data may include information such as an indication that a user pressed the letter “A” at 2:51:00 PM and/or an indication that a user pressed a first character, then a second character and the delay between pressing the two characters was 3 seconds.

The keyboard application 212 may collect timestamps of when each key on the keyboard application 212 is pressed, time between each keypress, number of backspace usages, number of autocorrect occurrences, and so on as keystroke dynamic data. In some examples, the keyboard application 212 also categorizes keystrokes by transition type, e.g., character-character, character-backspace, character-symbol, character-space, character-enter, alphanumeric-alphanumeric, alphanumeric-punctuation, and so on as keystroke dynamic data. Such a scheme allows for keystrokes to be categorized while maintaining the anonymity of the actual text being entered.

In some examples, the keyboard application 212 collects the keystroke dynamic data and sends the keystroke dynamic data to a database, such as a database of memory 110. Using the collected data, the keyboard application 212 computes and/or updates statistics. In this manner, the keyboard application 212 maintains the statistics on a rolling basis, which allows for both real-time feedback and observation of trends.

For example, the keyboard application 212 may analyze initial keystroke dynamic data for statistics (e.g., quantile estimates, reservoir sampling, P² algorithm, and so on). The keyboard application 212 may then send and store the initial keystroke dynamic data and any determined statistics associated with the initial keystroke dynamic data. Subsequently, the keyboard application 212 receives additional keystroke dynamic data, which prompts the keyboard application 212 to update the database with the additional keystroke dynamic data and update the statistics in view of the additional keystroke data.

FIG. 2B depicts analysis that may be done using keystroke dynamic data, in accordance with example embodiments. In particular, FIG. 2B includes a keystroke pattern chart 220, a typing variability chart 230, and an autocorrect rate chart 240. The keystroke pattern chart 220 depicts some example keystroke patterns of keystroke dynamic data, which are representative of keystrokes collected by the keyboard application 212 while a user is typing the note 214. The keystroke pattern chart 220 plots inter-key delay versus time. Analyzing inter-key delay (i.e., the delay between keystrokes) allows for insights into the user's behavior. For example, in the keystroke pattern chart 220, the inter-key delay between a keystroke to hit the last letter of a line and a keystroke to hit enter may be considerably less than the inter-key delay between hitting enter and the first letter of the line. This pattern may be expected, since a user may contemplate for a length of time before continuing to enter items into the list in notes 214.

Additionally, as mentioned above, keystroke dynamic data may include keystroke patterns that have been categorized into different transition types. For example, the keystroke pattern chart 220 depicts various transition types including alphanumeric to alphanumeric, alphanumeric to backspace or backspace to backspace, alphanumeric to special character or special character to alphanumeric, alphanumeric to punctuation or punctuation to alphanumeric, autocorrect event, or a combination thereof. These transition types may be determined from the keystroke dynamic data collected by the keyboard application 212 and may contribute to determining one or more cognitive and/or physical characteristics of the user.

The typing variability chart 230 depicts an example distribution of variability among inter-key delays by plotting the number of occurrences per inter-key delay length along with statistical measurements derived from the inter-key delays. Namely, the typing variability chart 230 includes a 25^{th} percentile inter-key delay line 232, a median inter-key delay line 234, a 95^{th} percentile inter-key delay line 236, and a median absolute deviance inter-key delay line 238. These statistics may be obtained through analyzing keystroke dynamic data.

For example, to calculate the 25^{th} percentile, the keyboard application 212 may multiply 0.25 by the number of inter-key delay samples and determine, in an ordered list of inter-key delay samples, the inter-key delay sample at the resulting number. Similar calculations may be repeated for the median (50^{th} percentile inter-key delay) and the 95^{th} percentile inter-key delay. The median absolute deviance inter-key delay may be obtained through determining the median inter-key delay, calculating the deviations of each inter-key delay from the median inter-key delay value, and taking the median of those calculated deviations. Other statistics are also possible.

The autocorrect rate chart 240 depicts an example of an autocorrect rate among samples of keystroke dynamic data, represented as a bar indicating the number of characters typed and another bar indicating the number of characters that were autocorrected. Particularly, for devices with smaller keys and/or smaller keyboards, mistakes may be common and a software on the device may automatically correct for any mistakes that the user may make. For example, “lettuce” may be autocorrected to “lettuce,” “carrots” may be autocorrected to “carrots,” and so on. These autocorrected letters may be counted and included in the autocorrect rate chart 240 as the second column.

After the keyboard application 212 collects keystroke dynamic data, the keyboard application 212 may also update any statistics related to the keystroke dynamic data. For example, upon registering an additional keypress, the keyboard application 212 may update the keystroke pattern chart 220 with additional data points. For example, if the keyboard application 212 receives an indication that the user pressed one or more additional keys, then keyboard application 212 may (1) determine the delay between the additional keypress and the previous keypress and (2) update the appropriate column in the typing variability chart 230. And based on the delay between the additional keypress and the previous keypress, the calculations associated with the 25^{th} percentile inter-key delay line 232, the median inter-key delay line 234, the 95^{th} percentile inter-key delay line 236, and the median absolute deviance inter-key delay line 238 may also be updated as well. Further, if the additional keypress caused one or more characters, words, sentences, etc. to be autocorrected, then the autocorrect rate chart 240 may also be updated. Other charts, statistics, and/or models may also be updated.

In some examples, after a length of time, a neurological disorder, other mental disorder, a physical disorder, other disorder, or a change in behavior of a user may alter typing patterns. As an example, the user of the computing device 100 may change their behavior, e.g., a user with bipolar disorder changes from an episode of mania to an episode of depression, causing the updated keystroke dynamic data to be fairly different from the previously collected keystroke dynamic data.

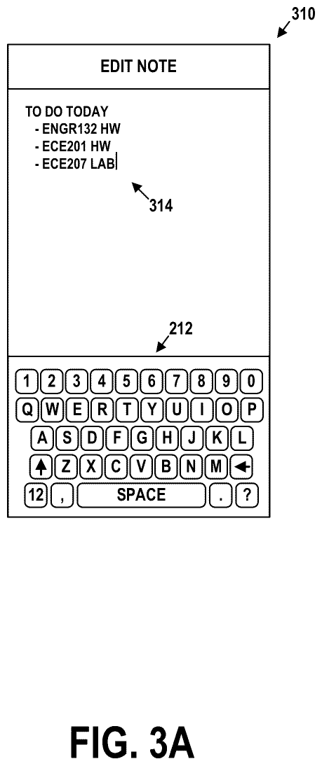

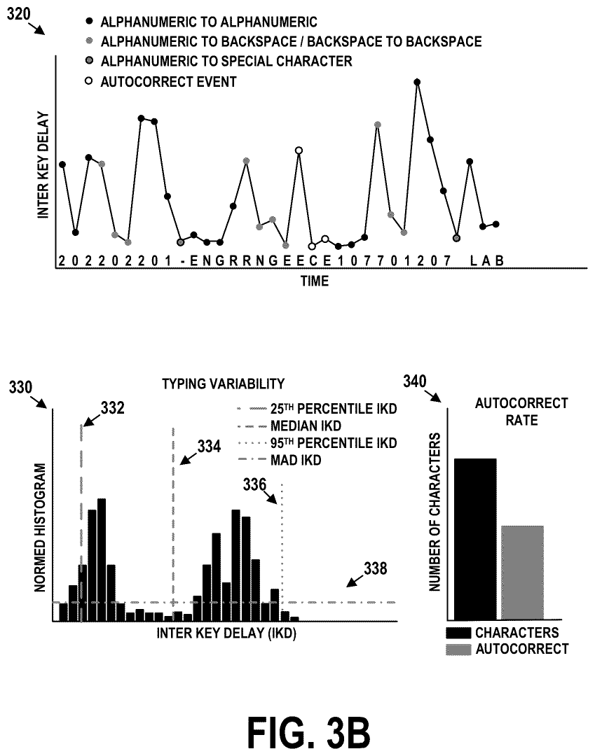

For example, FIG. 3A depicts a user interface 316 of the computing device 100 collecting additional digital behaviorome data, in accordance with example embodiments. The additional digital behaviorome data may be passively collected as a user of the computing device 100 is using the computing device 100. A user using the computing device 100 may use the keyboard application 212 to type out a note 314. The keyboard application 212 collects keystroke dynamic data on the user's typing patterns.

FIG. 3B depicts additional data collected by the computing device 100, in accordance with example embodiments. FIG. 3B includes an updated keystroke pattern chart 320, an updated typing variability chart 330, and an updated autocorrect rate chart. From the updated keystroke pattern chart 320, it may be observed that the inter-key delays between keystrokes may be higher than before (e.g., higher than data collected a day before, for example, data depicted in FIG. 2B), the difference being more apparent in the updated typing variability chart 330. The updated typing variability chart 330 has a bimodal distribution as shown, which demonstrates the increase in duration of inter-key delays in comparison to typing the variability chart 330. Further, the updated typing variability chart 330 include updated statistics, as indicated by an updated 25^{th} percentile inter-key delay line 332, an updated median inter-key delay line 334, an updated 95^{th} percentile inter-key delay line 336, and an updated median absolute deviance inter-key delay line 338. It may be observed that the inter-key delay lines (i.e., the updated median inter-key delay line 334, the updated 95^{th} percentile inter-key delay line 336, and the updated median absolute deviance inter-key delay line 338) are all shifted to the left, which may be due to the increase in number of samples having a higher inter-key delay. Additionally, the updated autocorrect rate chart 340 may indicate a higher number of samples having been updated.

Through the analysis of keystroke dynamic data as indicated by the charts in FIGS. 2B and 3B, the computing device 100 may conclude that the user of computing device 100 may have changed concentration levels, such as by becoming more distracted or by some other factor. Additionally or alternatively, the changes may be indicative of a broader underlying issue, such as a neurological and/or physical condition.

As mentioned above, other sources of data may also be collected from the user's mobile device (e.g., the computing device 100). For example, the keyboard application 212 (or other application on the computing device 100) may collect and store data regarding gestures registered on a touchscreen of the computing device 100, user movements collected from a gyroscope and/or accelerometer of the computing device 100, GPS signals from a sensor of the computing device 100, and so on. Keystroke dynamic data collected by the keyboard application 212 (or another application on the computing device 100) as well as the other examples of data mentioned above (e.g., the data from one or more sensors on a computing device including data from gyroscopes, accelerometers, touchscreens, and so on) may be collectively referred herein as digital behaviorome data. The digital behaviorome data may be analyzed collectively to extract user behavior patterns and be used to detect any underlying neurological and/or physical disorders.

### IV. Example Analysis Methods

To assess overall cognitive function, the computing device 100 may apply unsupervised machine learning methods on the obtained digital behaviorome data. Some unsupervised machine learning methods include regression analyses, unsupervised low-dimensional embedding, latent variable inference models (e.g., HMMs), clustering methods, a combination thereof, and/or other unsupervised machine learning methods. In some examples, the computing device 100 determines one or more user baseline models. Each of the user baseline models may include statistical relationships between at least two user interaction features, and each of the user baseline models corresponds to one or more physical, emotional, or cognitive user characteristics.

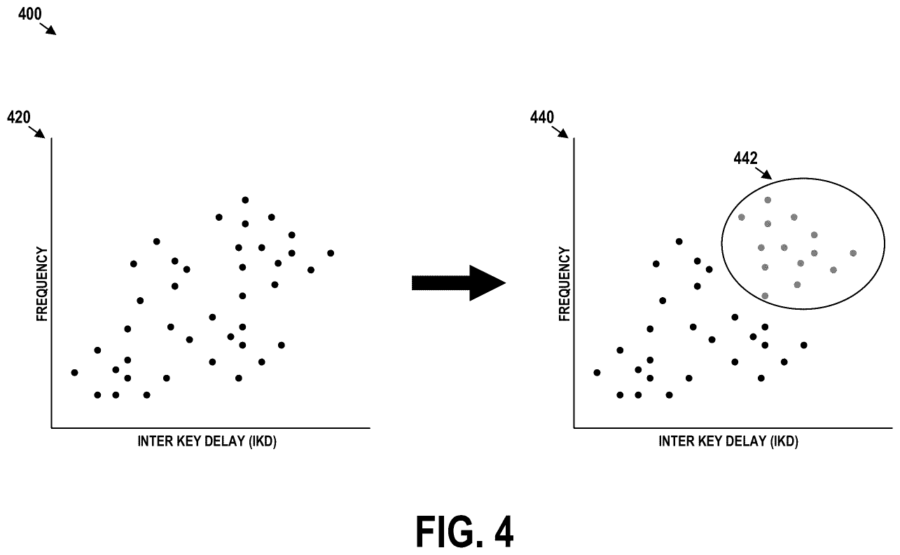

For example, FIG. 4 depicts a user baseline model 400, in accordance with example embodiments. The user baseline model 400 may involve clustering digital behaviorome data into different groups such that each data point of a group may correspond to whether a particular physical, emotional, or cognitive user characteristic is within an expected range. For example, the user baseline model 400 includes a chart 420, which illustrates the relationship between two user interaction features (i.e., inter-key delay and frequency) for a particular user over various periods of time (e.g., frequency per day over the period of a few months).

Whether the particular physical, emotional, or cognitive user characteristic is within the expected range for the user may be obtained through mathematical and/or visual analysis of the chart 420. For example, the user baseline model 400 also includes a chart 440, which illustrates the same relationship between inter-key delay and frequency for the same particular user over a period of time (e.g., Monday mornings). Here the data has been analyzed (visually or mathematically) to indicate a cluster 442 of abnormal data points. During the few months over which the digital behaviorome data was collected, the user may have had inattentiveness indicating a broader underlying neurological and/or physical disorder, and this inattentiveness may be indicated by cluster 442. Outside of the cluster 442, digital behaviorome data that displays relatively high frequency inter-key delays with relatively long inter-key delays may be simply indicative of lack of attention that the user may have.

In some examples, the inattentiveness might not be entirely atypical (e.g., not indicative of a broader underlying neurological and/or physical disorder) unless the inattentiveness continues for a length of time. Thus, with the collection of additional digital behaviorome data, the keyboard application 212 (or other application on computing device 100) may analyze the data to see where the additional digital behaviorome data falls within cluster 442. And if a significant number of points analyzed from the digital behaviorome data do fall within cluster 442, then the keyboard application 212 (or another application on the computing device 100) may notify the user of the computing device 100 of the abnormal samples in the digital behaviorome data. In some examples, the significant number of data points indicating an abnormality may be determined through a statistical test, e.g., a Student t-test, analysis of variance (ANOVA), among many other examples.

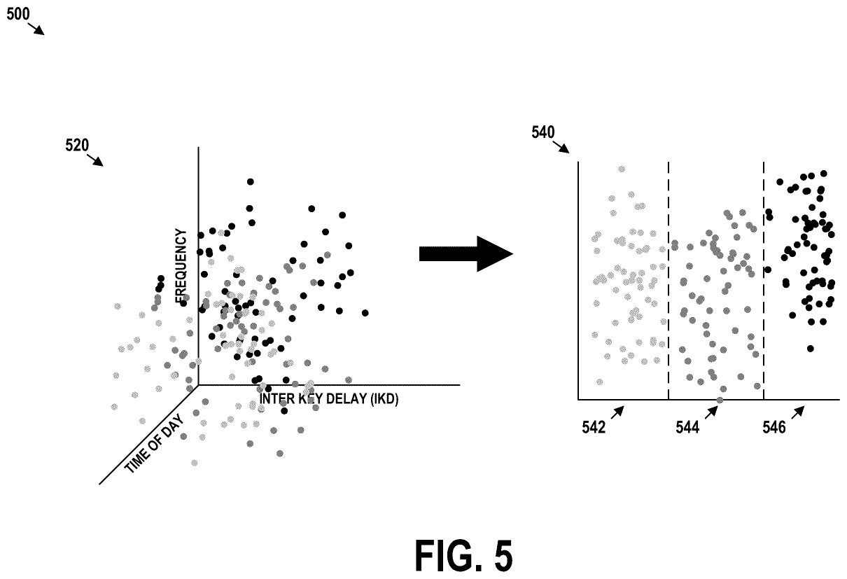

High-dimensional and unsupervised machine learning methods may be applied to the data to reduce the dimension before analyzing the data in the method described above. For example, FIG. 5 depicts a user baseline model 500, in accordance with example embodiments. The user baseline model 500 may include a chart 520 and a chart 540. The chart 520 may plot the relationship of three user interaction features, including inter-key delay, frequency, and time of day. In comparison to the digital behaviorome data plotted in the chart 420 and the chart 440 of the user baseline model 400, the digital behaviorome data plotted in the chart 520 may be more difficult to cluster visually due to the dimensionality of the data.

The dimensionality of the digital behaviorome data plotted in the chart 520 may be reduced in a variety of ways, such as through linear methods (e.g., principal component analysis (PCA), support vector machine (SVM), and so on) and through non-linear methods (e.g., through kernelization of linear projection methods, uniform manifold approximation and projection (U-map), t-distributed scholastic neighbor embedding (t-SNE), among other non-linear methods).

The chart 540 plots the digital behaviorome data of the chart 520 after the dimension has been reduced. It may be observed that, after reducing the dimensionality of the plotted keystroke dynamic data, groups of data may be clearly observed from the chart 540. These groups of data may then be analyzed in a manner similar to that of the chart 420 and the chart 440 of the user baseline model 400. For example, each group in the chart 540 may indicate a certain property of a user that may or might not indicate an underlying neurological and/or physical disorder. For example, a group 542, a group 544, and a group 546 may indicate progressions of a disorder through time, such that keyboard usage patterns of a user is in the group 542 when there is no indication of an underlying disorder, keyboard usage patterns of the user is in the group 544 when there is slight indication of an underlying disorder, and keyboard usage patterns of the user is in group 546 when a disorder has progressed significantly.

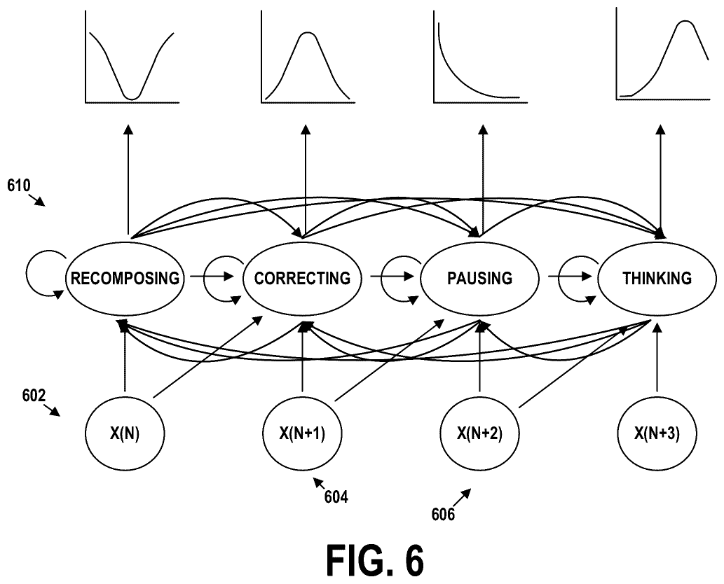

FIG. 6 depicts a user baseline model 600, in accordance with example embodiments. The user baseline model 600 may be used to classify digital behaviorome data in categories, such as digital behaviorome data collected when a user is composing, correcting, recomposing, pausing, waiting for others to respond, becoming distracted, thinking about what to type next, or a combination thereof, among other examples. The categories may then be used to determine a baseline model (e.g., what digital behaviorome data is anticipated to resemble under normal cognitive and physical user function). Significant deviations from the baseline model may be indicative of a broader underlying disorder.

The user baseline model 600 may use a Hidden Markov Model (HMM) 610 to classify digital behaviorome data in categories of cognitive processes. Other probabilistic graphical models may also be used. The HMM 610 may take sequential data 602 (e.g., having the same time axis) as an input. Each entry of the sequential data 602 may be representative of user interaction features from sequential periods in time. For example, x(n) may be representative of the amount of inter-key delay between two keystrokes, x(n+1) may be representative of the amount of inter-key delay between two subsequent keystrokes, and so on. As another example, x(n) may be representative of statistics (e.g., 25th percentile inter-key delay, median inter-key delay, 95th percentile inter-key delay, median absolute deviance inter-key delay, autocorrect delay, or a combination thereof) collected during a period in time, x(n+1) may be representative of those user interaction features collected during a subsequent point in time, among other examples.

The keyboard application 212 may use the HMM 610 to predict latent variables 604. The HMM 610 may include various parameters that represent probabilities of transitioning from a sample or samples of sequential data 602 to the latent variables 604. For example, given a certain x(n), a user that caused the statistics of x(n) may have a 0.2 probability of being in the midst of recomposing a text, a 0.4 probability of being in the midst of correcting a text, 0.1 probability of being in the midst of pausing, and 0.3 probability of being in the midst of thinking about what to type.

Further, the HMM 610 may include transition probabilities between latent variables. For example, assuming that the user is in the midst of recomposing a text, there may be a 0.2 probability of continuing to recompose that text, 0.3 probability of correcting the text, 0.2 probability of pausing in correcting the text, and 0.3 probability of thinking about correcting the text. These various parameters of HMM 610 may be determined through variational inference.

Latent variables 604 may be representative of cognitive processes occurring during the entry of sequential data 602. The keyboard application 212 might not be able to directly observe the latent variables 604, but may instead deduce the latent variables from the sequential data 602. The latent variables 604 may also be sequential such that each predicted latent variable depends on the previous latent variable.

For example, a user “recomposing” a message may be most likely to be “correcting” a message next, and a user “correcting” a message may be most likely to subsequently “pause” in typing a message, and so on. Other latent variables are also possible. For example, possible latent variables may further include waiting for the other person to respond, becoming distracted, among other possible cognitive processes.

Once latent variables 604 are predicted from the sequential data 602, the keyboard application 212 may model timing dynamics 606 associated with cognitive processes represented by the latent variables 604. the timing dynamics 606 represent distributions of inter-key delays that are associated with the cognitive processes represented by the latent variables 604, as depicted by the typing variability chart 230 and the typing variability chart 330 of FIGS. 2B and 3B, respectively. For example, the cognitive process of pausing may be associated with a timing dynamic that is modeled as a power-law or a log-normal distribution that displays scale invariance across a time-scale range of naturalistic human behaviors.

The parameters associated with these distributions in timing dynamics 606 may be determined through maximum likelihood estimation approximation. The computing device 100 may collect additional digital behaviorome data to be inputted into the HMM 610 to determine a specific timing dynamic of the timing dynamics 606, and the collected additional digital behaviorome data may be compared with the determined specific timing dynamic to determine whether a physical, emotional, or cognitive user characteristic for the user is within an expected range.

In some examples, determining one or more user baseline models involves determining a mood stability user baseline model. To determine a mood stability user baseline model, the computing device 100 may determine a variability between the plurality of user interaction features for a period of time. Based on the variability between the plurality of user interaction features, the computing device 100 may also determine a threshold deviation from the variability associated with expected mood stability during the period of time, where the threshold deviation may be determined from a percentile calculation of the variability.

In some examples, the user interaction features may include a backspace usage feature, an input mistakes feature, and an input time feature. Determining one or more user baseline models may include determining an impulsivity user baseline model. The impulsivity user baseline model is similar to the user baseline model 500 but includes relationships between a backspace usage feature, an input mistakes feature, and an input time feature.

To determine the impulsivity baseline model, the computing device 100 may determine a lower-dimensional projection of these features (e.g., from the chart 520 to the chart 540). Based on this lower-dimensional projection, the computing device 100 determines a low impulsivity time range associated with a low impulsivity user characteristic. Differences between an input time value associated with the input mistakes feature and a further input time value associated with the backspace usage feature falling within the low impulsivity time range indicate that the user is associated with a low impulsivity user characteristic. The impulsivity user baseline model may include the low impulsivity time range, and the statistical value may be based on values of at least two of the additional user interaction features relative to the low impulsivity time range. Also based on this lower-dimensional projection, the computing device 100 may determine a high impulsivity time range associated with a high impulsivity user characteristic, where the differences between an input time value associated with the input mistakes feature and a further input time associated with the backspace feature falling within the high impulsivity time range indicates that the user is associated with a high impulsivity user characteristic.

A similar method may be used to determine low and high attention ranges. In particular, the one or more user baseline models may include an attention user baseline model, and the user baseline model may include relationships between a backspace usage feature, an input mistakes feature, and an input time feature. The computing device 100 may determine a lower-dimensional projection of these three features. Based on this lower-dimensional projection of these three features, the computing device 100 may determine a low attention range that is associated with a low attention user characteristic. The computing device 100 may collect additional digital behaviorome data corresponding to additional user interaction features, and the statistical value may then be based on values of the additional user interaction features relative to the low attention range.

In some examples, the user baseline model may be a processing speed model that includes relationships between a typing rhythm feature and an accuracy feature. The processing speed model may be based on historical processing speed values (e.g., processing speed values for the previous week, previous month, previous year, etc.). Based on the processing speed model, the computing device 100 may determine a predicted processing speed value for a period of time (e.g., a day, an hour, an afternoon, etc.). The computing device 100 may compare the predicted processing speed value for the period of time with the processing speed value of the historical processing speed values. If the processing speed value is greater than the predicted processing speed value by outside the predefined range, then computing device 100 may determine that the user processing speed characteristic for the user is within the expected range.

Other models may also be used to determine whether the one or more physical, emotional, or cognitive user characteristics falls within the expected range.

In some examples, the computing device 100 may collect additional digital behaviorome data that includes a plurality of additional user interaction features, and the additional user interaction features may be used as a basis to select a particular user baseline model to use to determine whether the particular physical, emotional, or cognitive user characteristic is within an expected range. For example, the computing device 100 may collect and/or determine additional user behaviorome data (e.g., the additional data depicted in FIG. 3B) including user interaction features of frequency and inter-key delay and select between the three user baseline models illustrated in FIGS. 4-6.

Since FIG. 4 includes user interaction features including frequency and inter-key delay, the computing device 100 then selects the user baseline model 400 as the model to use to determine whether the additionally collected digital behaviorome data corresponds to inattentiveness. As another example, the computing device 100 may collect and/or determine additional user behaviorome data including user interaction features of frequency, time of day, and inter-key delay. Since FIG. 4 includes these user interaction features, the computing device 100 selects user baseline model 500 as the model to use to determine the progression of a disorder through time.

Additionally or alternatively, the computing device 100 may select the user baseline model based on the particular physical, emotional, or cognitive user characteristic that is being determined. For example, the computing device 100 may generate graphics to show the physical, emotional, and/or cognitive health of a user of the computing device 100. A particular graphic may display a user attention level, which may make use of whether the user is being inattentive. The computing device 100 may thus use user baseline model 400 as a basis to determine which user baseline model to use. Accordingly, the computing device 100 may use the needed physical, emotional, or cognitive user characteristic as a basis to determine which user baseline model to use.

In some examples, after selecting a particular user baseline model to use, the computing device 100 compares the values of the additional user interaction features to the particular user baseline model. For example, if user baseline model 400 is selected, the computing device 100 may analyze the additional frequency and additional inter-key delay values in the context of the clusters developed in the user baseline model 400. The additional frequency and additional inter-key delay values may be plotted to determine the number of points that fall within the cluster 442 as the statistical value.

As another example, if the user baseline model 500 is selected, the computing device 100 may analyze the additional frequency values, the additional time of day values, and the additional inter-key delay values in the context of the user baseline model 500. The additional frequency values, the additional time of day values, and the additional inter-key delay values may be plotted and compared with user baseline model 500 to determine a number of points that fall within a region of chart 540.

As a further example, if the user baseline model 600 is selected, the computing device 100 may analyze the additional digital behaviorome data in the context of the user baseline model 600. The additional digital behaviorome data may be inputted into the HMM 610 to determine a timing distribution, and the additional digital behaviorome data may be compared with the determined timing distribution using a statistical test, e.g., a Student t-test. A Student t-test may determine a p-value that corresponds with the significance of the difference between the determined timing distribution and the additional digital behaviorome data. In further examples, other suitable statistical tests may be used, and other statistical tests may use different measures to measure differences.

The computing device 100 may then determine whether the statistical value is outside a predefined range to indicate that the particular physical, emotional, or cognitive user characteristic associated with the particular user baseline model is within an expected range. For example, if the user baseline model 400 is selected, a predefined range of three to five data points falling within the cluster may be applied. If there are more than the predefined range of data points falling within the cluster (e.g., the number of data points outside the cluster may be outside the predefined range), then the user may be being inattentive. Whereas, if there are less than the threshold value of data points falling within the cluster (e.g., the number of data points outside the cluster may be outside the predefined range), the user may also be being inattentive.

As another example, if the user baseline model 500 is selected, a predefined range of three to five data points falling within a particular region may indicate that the user's disease progression is at a particular stage. If they are outside the predefined range of data points within a particular region, then the user's disease progression may not be in that particular stage. Whereas, if they are inside the predefined range of data points within the particular region, then the user's disease progression may be at that particular stage.

As a further example, if the user baseline 600 is selected and the significance of differences between the timing distribution and the additional digital behaviorome data is quantified through a Student t-test, then the predefined range corresponding to the p-value may be from zero to 0.05. If the p-value between the timing distribution and the additional digital behaviorome data is between zero and 0.05, then the difference is significant and the user may be determined to have different recomposing, correcting, pausing, thinking, etc. If the p-value between the timing distribution and the additional digital behaviorome data is outside of the range between zero and 0.05, then the difference is not significant, and the user may be determined to have not changed their recomposing, correcting, pausing, thinking, etc. typing patterns.

In each of the cases listed above, determining that the statistical value is above/outside the predefined range yields information relating to the particular physical, emotional, or cognitive user characteristic being within an expected range (e.g., a range that is indicative of a particular state of the particular physical, emotional, or cognitive user characteristic). In some examples, the predefined range may instead be a threshold value such that a statistical value above and/or below the threshold value may be indicative of a particular state of the particular physical, emotional, or cognitive characteristic.

In some examples, these classifiers may be unsupervised machine learning models that do not involve the use of a learning function with individual weights to be manipulated and optimized in order to minimize a loss function.

Further, in some instances, the digital behaviorome data comprising user interaction features may include one or more gestures or gesture-related characteristics, such as left-right or right-left swiping of the keyboard, left-right or right-left swiping of the screen, various tapping gestures, the pressure of the input onto the user interface, the velocity and linear/angular acceleration that the user swipes or otherwise interacts with the user interface, the spatial distribution and variability of the pixels traversed during gesture inputs the spatial distribution of the optimal path of the intended texts, pauses between consecutive gesture inputs, and/or the transition between gesture inputs, typing, and the use of backspaces or autocorrection/autosuggestion, among other examples. This digital behaviorome data may be collected by a touchscreen of the computing device 100 while computing device 100 is concurrently collecting gyroscope and/or accelerometer data. In some examples, computing device 100 may also be concurrently collecting other data, including global positioning system (GPS) data, phone activity data, etc.

In some examples, linguistic features of text entered from a keyboard application (e.g., the keyboard application 212) may also be included in digital behaviorome data. These linguistic features may include phonological features, morphological features, and/or semantic features, as well as other like features. The text may be entered as part of a chatbot conversation or within a messaging system between a user and their healthcare provider.

Natural language processing algorithms (e.g., word embedding and sentiment analysis) may be applied to these features of the digital behaviorome data to passively infer cognitive domains related to language functioning. In some examples, these natural language processing algorithms may be implemented on a server device. For example, the digital behaviorome data may be sent to the server device and the server device may apply the natural language processing model. The result (e.g., the user's physical, emotional, and/or cognitive user characteristic) may be sent back to the computing device 100 for display. A differential privacy algorithm may be used to further protect data security and user confidentiality.

In some examples, the processes described herein might not involve a benchmark test. For example, the user baseline model and/or the user's physical, emotional, and/or cognitive user characteristics might not be compared to a neuropsychological benchmark test such that the platform does not compare the user against a tested standard.

### V. Example User Interfaces

After having determined that the particular physical, emotional, or cognitive user characteristic for the user is within an expected range, the computing device 100 may display an indication that the particular physical, emotional, or cognitive user characteristic for the user is within the expected range. Additionally or alternatively, the computing device 100 may display an interpretation related to the statistical value for the particular physical, emotional, or cognitive user characteristic based on the comparison of values from the at least two of the additional user interaction features relative to the particular user baseline model. Other examples are possible as well.

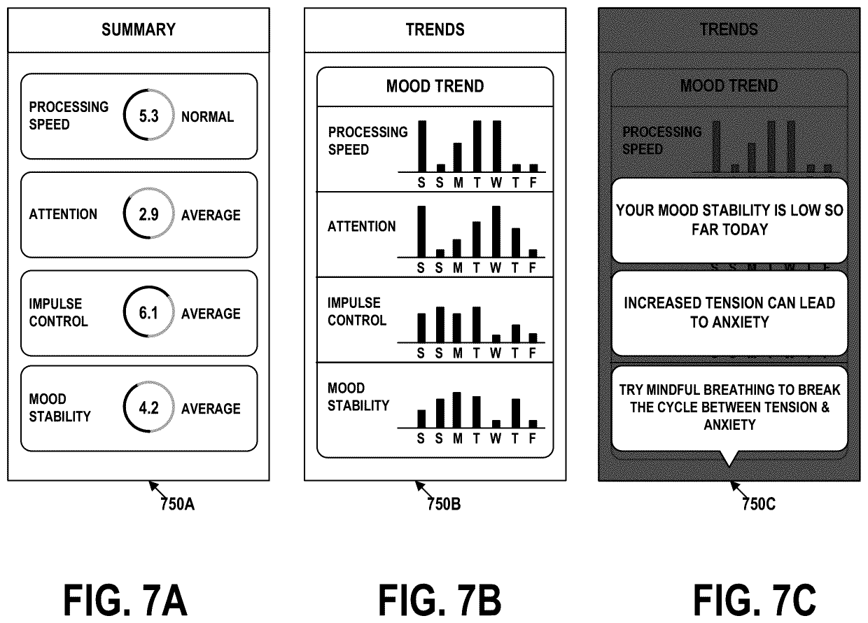

To illustrate, FIGS. 7A, 7B, and 7C depict a user interface 750a, a user interface 750b, and a user interface 750c, respectively, in accordance with example technologies. Each of the user interface 750a, the user interface 750b, and the user interface 750c may be used to convey various information on the determined physical, emotional, and/or cognitive user characteristics. In some examples, the user interface 750a, the user interface 750b, and/or the user interface 750c may be displayed in real time, and these user interfaces may be presented at the momentary, daily, weekly, and monthly level with customizable alerts notifying the user (or other contact, as will be discussed later) of deviations from regular and/or average performance.

For example, referring to FIG. 7A, the user interface 750a depicts a summary of cognitive health, including metrics on processing speed, attention, impulse control, and mood stability. Each of these physical, emotional, and/or cognitive user characteristics (e.g., processing speed, attention, impulse control, and mood stability) may be determined using the methods and processes described above. In response to a change in value in a physical, emotional, and/or cognitive user characteristic, the computing device 100 may update the user interface 750a with the updated physical, emotional, and/or cognitive user characteristic.

In some examples, these values corresponding to the physical, emotional, and/or cognitive user characteristics may be plotted over time. For example, in FIG. 7B, the user interface 750b depicts processing speed, attention, impulse control, and mood stability over time. These trends may be updated periodically, such as on example frequencies of every day, every few hours, etc., as new data is being collected and analyzed. If a new value corresponding to the physical, emotional, and/or cognitive user characteristic deviates significantly from what is expected and/or when the deviation occurs for an extended period of time, the computing device 100 may generate and display an alert to the user that the value/trend of the physical, emotional, and/or cognitive user characteristic seems to be abnormal.

In some examples, these trends and the value of the physical, emotional, and/or cognitive user characteristic are analyzed and improvements may be given. For example, as shown in FIG. 7C, the user interface 750c depicts improvements that may be given to a user with low mood stability, including a suggestion that “increased tension can lead to anxiety” and “try mindful breathing to break the cycle between tension & anxiety.” These prompts may be customized based on what the user has seen in the prompts previously, other trends in physical, emotional, and/or cognitive user characteristics, and values of physical, emotional, and/or cognitive user characteristics. In some examples, these prompts may be implemented as tips and nudges such that the computing device 100 notifies the user of these tips every now and then (e.g., every day).

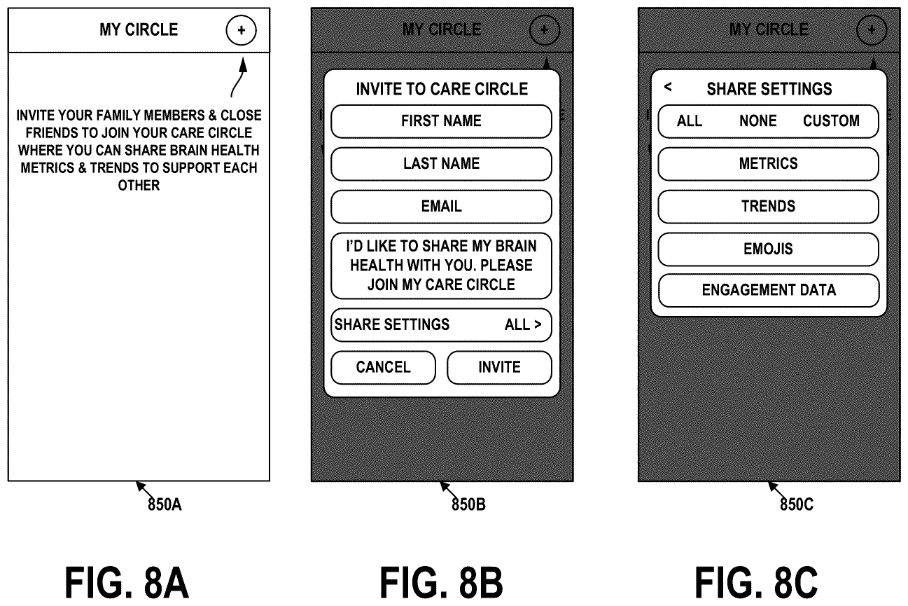

FIGS. 8A, 8B, and 8C depict a user interface 850a, a user interface 850b, and a user interface 850c, respectively, in accordance with example technologies. The user interface 850a, the user interface 850b, and the user interface 850c depict the process of adding a contact to the user's care circle and sharing physical, emotional, and/or cognitive user characteristics with contacts in the user's care circle. For example, in FIG. 8A, the user interface 850a depicts inviting contacts (e.g., family members and close friends) to join the user's chare circle such that the user can share their physical, emotional, and/or cognitive user characteristics (e.g., share the values and/or trends of the physical, emotional, and/or cognitive user characteristics depicted in the user interface 750a and the user interface 750b). In FIG. 8B, the user interface 850b depicts adding in a contact's information to invite them to view the user's physical, emotional, and/or cognitive user characteristics. And, in FIG. 8C, the user interface 850c depicts a user interface where the user may set the specific information to share (e.g., all, none, custom, values, trends, emojis, engagement data, etc.).

Having a care circle may be particularly useful if the user has physical, emotional, and/or cognitive disorders so that a contact may monitor them remotely. Further, a healthcare provider or researcher may be added as a contact in a care circle so that the healthcare provider or researcher may have access to the user's data. Such data access may facilitate informed treatment decisions based on up-to-date results on how the current treatment is progressing.

### VI. Example Keyboard Feedback

Example technologies may involve providing feedback on physical, emotional, and/or cognitive user characteristics directly within the digital behavior keyboard. By providing the feedback directly in the digital behavior keyboard itself, a user may be provided an indication of their cognitive functioning whenever they use their keyboard. There is no need for the user to open another application and view their cognitive feedback, which they might be less likely to do when experiencing a physical, emotional and/or cognitive disorder.

Existing health status apps often require use of a health app. It may be helpful for the user to view their health status while they are typing a text message to a friend. As such, by exiting the text messaging app and launching the health app, the user is then able to view and interact with the desired health data. However, it is then necessary to leave the health app and re-enter the text messaging app to pick up where they left off.

This user experience is problematic. It is inconvenient, requiring multiple taps or gestures to navigate between various apps. It is also prone to errors, such as forgetting to navigate back to the original app or disrupting the flow of thought by forcing abrupt changes in user interface models of the two apps. Additionally, reliance on memory of information may lead to errors and frustration from the user, especially in people with memory impairments or “brain fog.” And the process is time consuming. If the user just needs to check one piece of information, it is much more efficient and convenient to view that information immediately from the keyboard they are using, or with one tap on a designated icon residing on the keyboard itself.

An application (commonly referred to as an “app”) may add a digital behavior keyboard as an additional or alternative keyboard for the computing device 100 (FIG. 1). For instance, a user may use an application distribution service (i.e., an app store), such as the Apple App Store®, the Google Play Store® or the Microsoft Store®, to download an application that includes the digital behavior keyboard. Installation of the application may then add the digital behavior keyboard as an additional or alternative keyboard for the computing device 100.

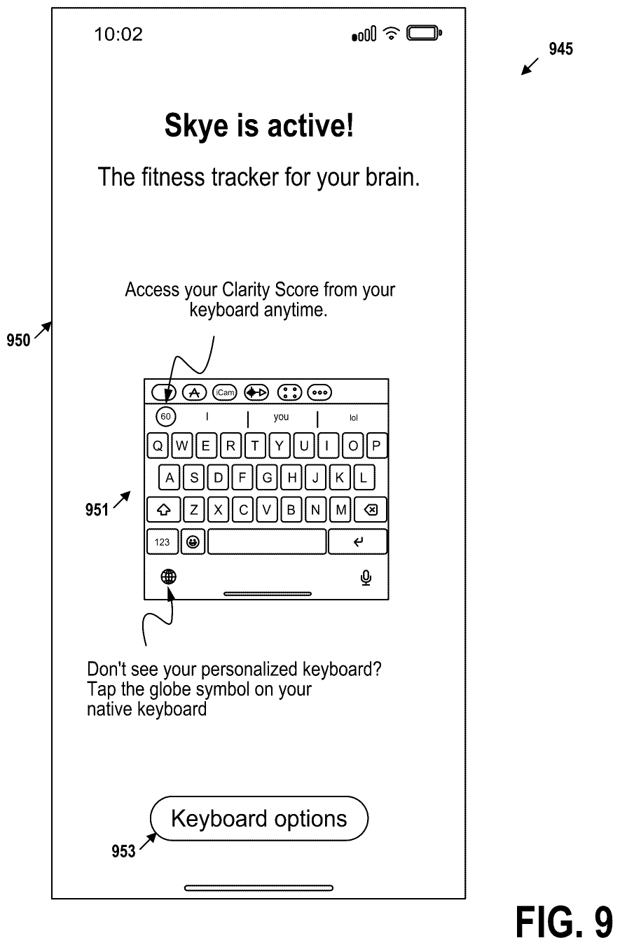

By way of illustration, FIG. 9 shows a user interface 950 of an example application 945 (the Skye application), which is configured to add an example digital behavior keyboard to an operating system. The user interface 950 may be shown to a user after installation and execution of the application 945 so as to provide some information regarding the digital behavior keyboard added by the app. As shown, the user interface 950 includes a region 951 that includes graphics and text to indicate to the user that a digital behavior keyboard is installed, and that the keyboard provides feedback on the user's cognitive state (here, referred to as a “Clarity Score”).

Also shown in the region 951 is an instruction on how to switch to use of the digital behavior keyboard (if not already enabled), as well as a selectable settings control 953 to access settings associated with the digital behavior keyboard (e.g., color and style). Example operating systems such as Apple iOS® or Android® permit a user to select among multiple keyboards. As such, after being added as an available keyboard, a user may use a setting of the operating system to enable the digital behavior keyboard. When enabled, the digital behavior keyboard replaces the default operating system (or a previously enabled third-party keyboard), which is referred to as a digital application keyboard.

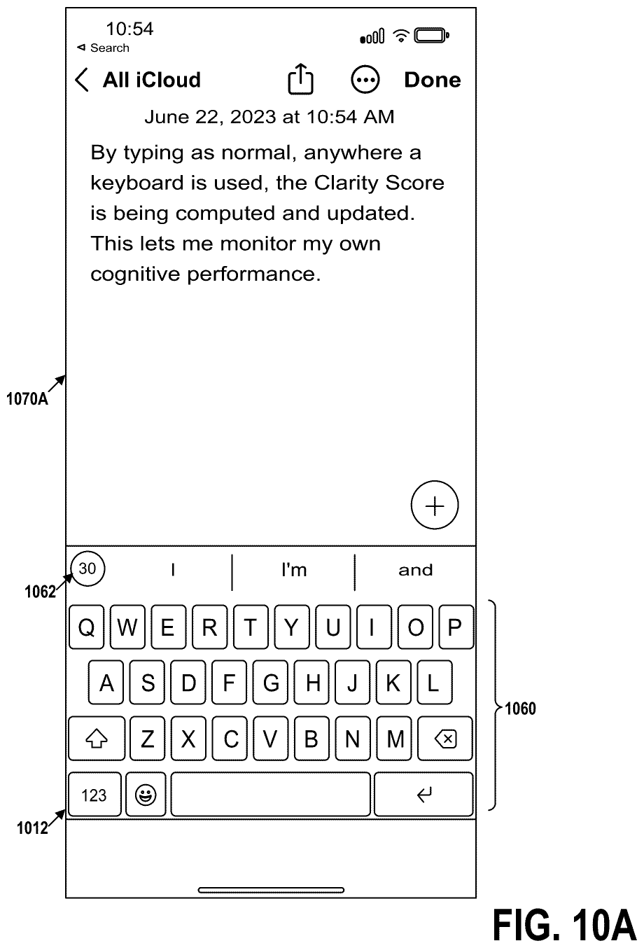

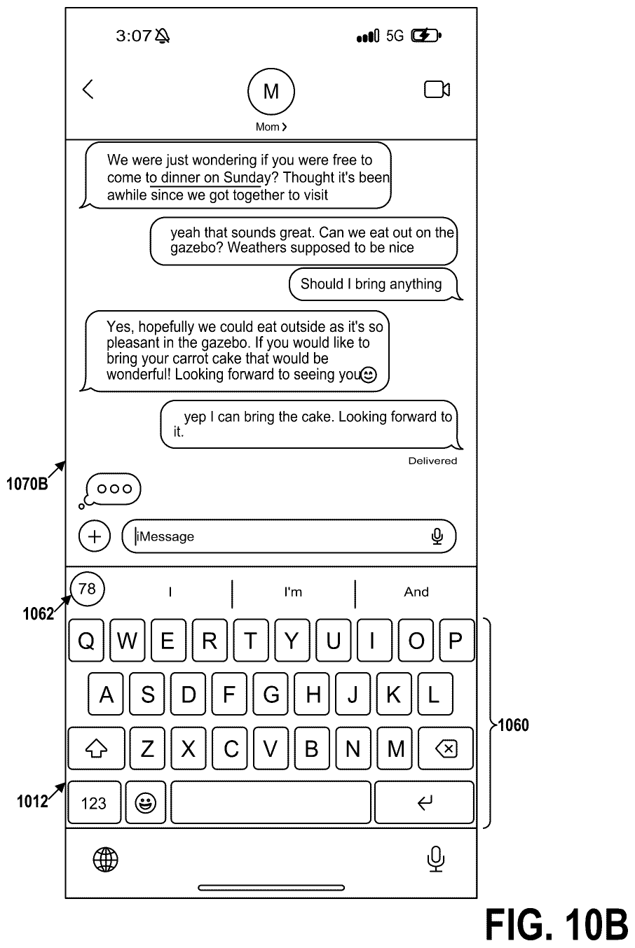

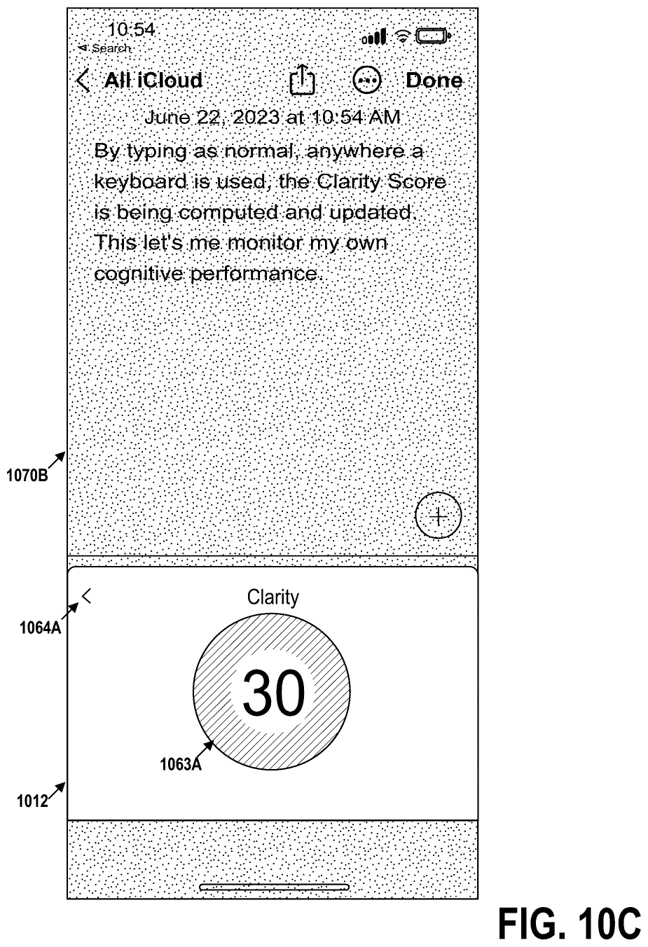

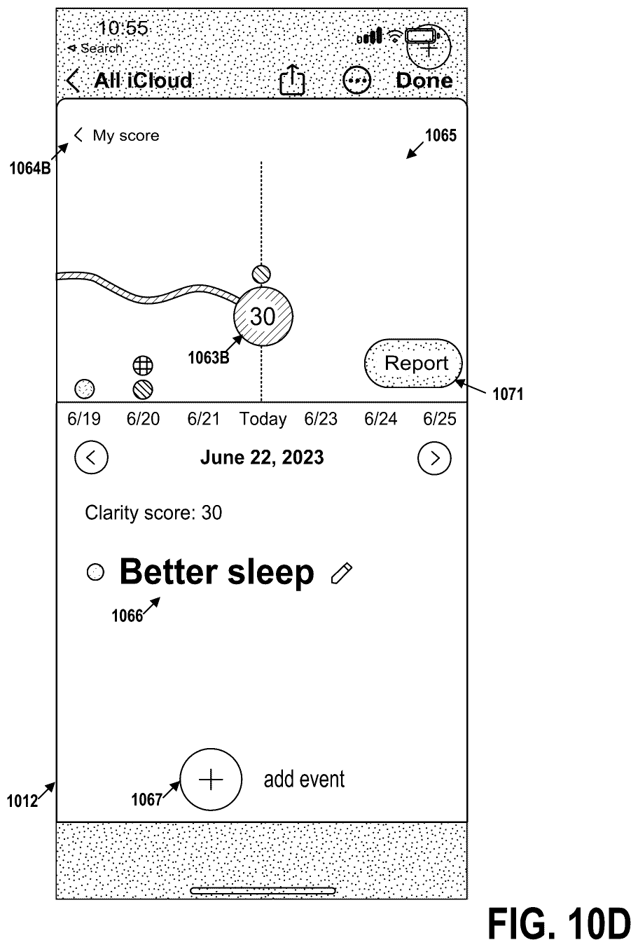

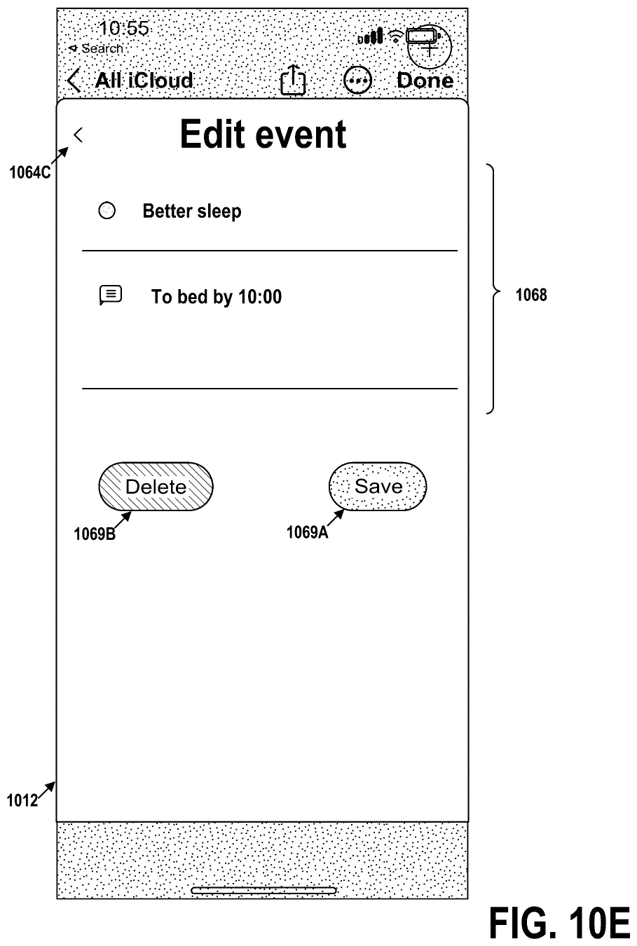

Similar to such digital application keyboards, the digital behavior keyboard includes keys that are selectable to provide input to various other applications or the operating system itself. By way of illustration, FIG. 10A shows an example digital behavior keyboard 1012, which may be an example of the keyboard application 212. The digital behavior keyboard may be installed by the application 945 on the computing device 100. The digital behavior keyboard 1012 includes keys 1060, which are selectable (e.g., via a touch input) to provide keystroke input to other applications on the computing device 100.

Here, the keys 1060 have been used to write an example note in a note taking application 1070A, as shown. The notetaking application 1070A is one example of many common apps that utilize keyboard functionality for textual entry, such as messaging apps, note-taking apps, email apps, and the like. In some cases, it is desirable to display cognitive feedback to the user without requiring the user to exit the current app (e.g., the notetaking application 1070A).

As also shown in FIG. 10A, the digital behavior keyboard 1012 includes an icon 1062. The icon 1062 shows a composite cognitive measure, which in this example is a quantitative value representing the user's cognitive state. Notably, the icon 1062 is displayed concurrently with the keys 1060 when the digital behavior keyboard 1012 is in use (i.e., displayed), which provides immediate feedback to the user regarding their cognitive state.

Example technologies include determining a composite cognitive measure based on the user keyboard usage patterns associated with processing speed and executive function that are described in the preceding sections. As noted previously, such a composite cognitive measure may be referred to herein as a Clarity Score. Within examples, the digital behavior keyboard 1012 may determine a composite cognitive measure and display the composite cognitive measure for the user.

More particularly, within certain examples, the composite cognitive measure represents a composite of metrics on processing speed, attention, impulse control, and mood stability. Such metrics are illustrated in the user interface 750A (FIG. 7A). Within examples, such metrics may be averaged, or otherwise combined so as to form a measure that is a composite of two or more metrics.

In various embodiments, dynamic keystroke data is used in determining the composite cognitive measure. Such dynamic keystroke data may include interkey delay data. The computing device 100 may record a time between successive keystrokes as the interkey delay data based on a first touchscreen input corresponding to a first keystroke and a second touchscreen input corresponding to a second keystroke. Such dynamic keystroke data may additionally or alternatively include autocorrect frequency (i.e., the frequency at which an autocorrect tool is invoked to correct a typographical error made while typing with the digital behavior keyboard 1012.

Within examples, the composite cognitive measure is based on at least autocorrect frequency and interkey delay. For instance, the composite cognitive measure may be based on a calculated mean of a sliding average of the autocorrect frequency and a median of the interkey delay measured over a designated interval. Such a metric provides an up-to-date measure as the user's cognitive state changes (as evidenced by their typing patterns) during use of the digital behavior keyboard 1012.

In some examples, the composite cognitive measure may be scaled or otherwise modified to provide the composite cognitive measure as a numerical value that is easy for a user to understand. For instance, the composite cognitive measure may be scaled such that it is represented as a linear integer ranging from 1 to 99. Yet further, the slope of the composite cognitive measure (c) may be modified such that the rate of increase (or geometric slope) of composite cognitive measure from 1 to 50 is greater that the slope of composite cognitive measure from 50 to 99, according to the following function ƒ(c):

Iƒc≤50:

ƒ(c)=1.5c

Else:

ƒ(c)=0.5c
Where: 0<c<100
   Other modifications, such as scaling to a different range, or conversion to a different scale (e.g., A-F letter grades), are contemplated as well.

As another example, FIG. 10B shows the keyboard 1012 being used to provide input to a text message app 1070B. Similar to FIG. 10A, the keyboard 1012 is shown with the keys 1060 and also an icon 1062 with the current compositive cognitive measure (here, “78”). Since text messaging is often one of the most used apps that involves keyboard text entry on mobile devices, being able to provide cognitive feedback directly on the keyboard 1012 concurrently with use of the text message app 1070B is potentially very helpful to the user.

In some examples, the keyboard 1012 may be configured to provide additional information, such as additional cognitive feedback, to the user via modifications to the keyboard 1012 while in use (i.e., while displayed). For instance, within examples, the icon 1062 is selectable to show additional cognitive feedback in place of the keys 1060. This additional cognitive feedback and/or a different representation of the compositive cognitive measure, among other examples.

To illustrate, FIG. 10C shows the keyboard 1012 after the icon 1062 has been selected. Here, the composite cognitive measure (here referred to as “Clarity” (i.e., Clarity Score)) is displayed as an icon 1063A, which is larger than the icon 1062. This increased size may be helpful to a user experiencing a cognitive issue, who may be struggling to comprehend the relatively smaller icon 1062. Although not shown in FIG. 10C, this view may additionally or alternatively include other information, such as a timeline of the Clarity Score throughout the day or week. To continue with text entry via the digital behavior keyboard 1012, the back icon 1064A is selectable to show the keys 1060.

In some examples, the icon 1063A is selectable to show yet further information. For example, FIG. 10D shows the keyboard 1012 after the icon 1063A is selected. Here, the screen area of the keyboard 1012 has been expanded to show a timeline region 1065. The timeline region 1065 includes a timeline of the composite cognitive measure by day as well as a larger icon 1063B showing the composite cognitive measure for the current day. This timeline allows the user to see trends in their cognitive ability over time.

As further shown in FIG. 10D, the keyboard 1012 provides an event region 1066. A user may use this area to view specific notes or events on each date, which may help contextualize their compositive cognitive score. Further, the user may set goals, such as better sleep or to exercise using this feature, which may improve their compositive cognitive score in the future. The event region 1066 includes an icon 1067, which is selectable to add an event. Events added for a particular date are shown in the timeline region 1065, as shown. To continue with text entry via the digital behavior keyboard 1012, the icon 1064B is selectable to go back to the previous view, or ultimately to show the keys 1060.

In FIG. 10D, the keyboard 1012 also includes a report icon 1071. When selected, the report icon 1071 generates an electronically-sharable report. The report may include data representing the composite cognitive measure over a pre-determined period of time along with related timeline information (e.g., on a graph). Electronic sharing of the report may be pre-configured (e.g., to share with a designated medical professional via the Internet) or configured via settings (e.g., accessed via the settings control 953 of the application 945).

To illustrate addition of an event or note, FIG. 10E shows the keyboard 1012 after the icon 1067 is selected. Here, the screen area of the keyboard 1012 has been modified to show a event editing region 1068. The event editing region 1068 includes areas for entry of an event title and note. Here, the example event title is “Better sleep” and the note is “To bed by 10:00”. Example events include (but are not limited to) health status, travel, stress, travel, diet, exercise, or medication schedules. After entry of a title and/or note, the user can save the event by selecting the icon 1069A or delete the event by selecting the icon 1069B, as shown. To continue with text entry via the digital behavior keyboard 1012, the icon 1064C is selectable to go back to the previous view, or ultimately to show the keys 1060.

As illustrated by FIGS. 10A-10E, the capability to display and enter information is integrated within the keyboard module itself, such that as the digital behavior keyboard is utilized by the user, custom programming code can update a graphic within the physical area of the digital behavior keyboard 1012 itself. Further capability may be provided that allows the user to interact with one or more graphic elements on the digital behavior keyboard 1012, to access additional information, or to initiate new actions such as place markers on a timeline. While undertaking these actions, the user can return quickly to the prior use of the digital behavior keyboard 1012, within the prior context of whatever activity the user was undertaking prior to interacting with the integrated icon.

Within examples, the digital behavior keyboard 1012 may be navigable via scroll and/or swipe gestures on the touchscreen display of the computing device 100. For instance, a user may swipe to show more of the timeline shown in the timeline region 1065 in FIG. 10D. As another example, a user may scroll to view additional events for a date (should the number of events not be displayable within the area provided on the digital behavior keyboard 1012. Other examples are possible as well.

In some examples, the digital behavior keyboard 1012 and/or the application may include an interface to enter a user identifier and/or a group identifier. A user identifier (e.g., a participant ID code) may uniquely identify the user across a group, or across all users. The group identifier associates the user with a particular group.

In some examples, comparisons of cognitive metrics such as the composite cognitive score may be made across group members may be made, which may facilitate evaluation of a user's cognition by the user or by a medical professional. In some examples, the user's data is sent to a computing device (e.g., in the cloud) which collates data from multiple users (e.g., the group or groups) and formulates the comparisons. Within examples, such data sharing is optional (involving user consent) and further is randomized to prevent identification of an individual user. In some examples, graphics and/or text showing the comparisons are displayed by the digital behavior keyboard 1012.

### VII. Example Methods

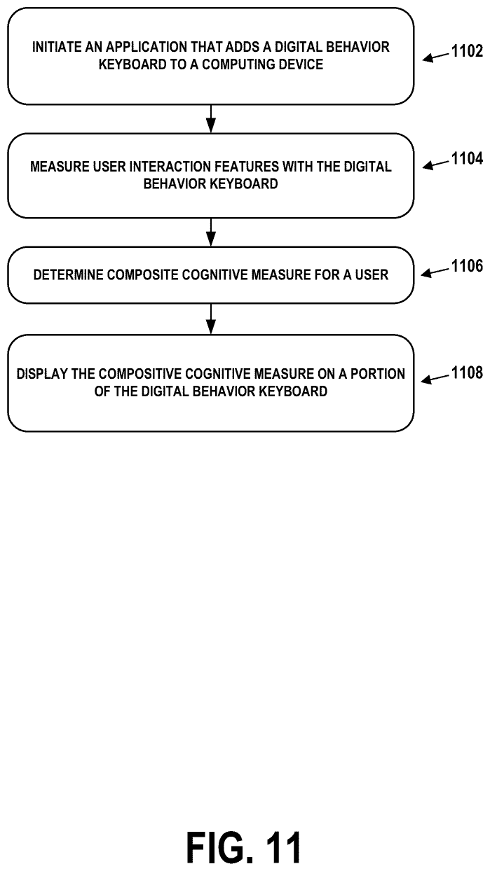

FIG. 11 is a flow chart of a method, in accordance with example embodiments. FIG. 11 may be implemented using computing device 100 or other suitable devices. In some examples, FIG. 11 may be implemented using one or more computing devices.

At block 1102, the method 1100 includes initializing an application that adds a digital behavior keyboard. For instance, the computing device 100 may initialize the application 945 (FIG. 9), which adds the digital behavior keyboard 1012 (FIGS. 10A-10E). When enabled, the digital behavior keyboard replaces a digital application keyboard on the computing device. For instance, the digital behavior keyboard 1012 may replace a default or third-party keyboard that was previously enabled on the computing device 100.

The digital behavior keyboard includes keys (e.g., the keys 1060) that are selectable via a touchscreen (e.g., the touch-screen 216) of the computing device 100 to provide input to other applications. Examples of the other applications include the note taking application 1070A and the text message application 1070B, among other applications described herein. Further examples include any application that is configured to take in text input.

At block 1104, the method 1100 includes measuring user interaction features with the digital behavior keyboard. For instance, the digital behavior keyboard 1012 may measure dynamic keystroke data representative of user keyboard usage patterns made while providing keystroke input via the keys to one or more of the other applications. The user dynamic keystroke data may include inter-key delay and autocorrect frequency as described in connection with section III. Within examples, measuring user interaction features with the digital behavior keyboard is performed while the digital behavior keyboard is enabled (i.e., active) on the computing device 100.

At block 1106, the method 1100 includes determining a composite cognitive measure for a user. For instance, the digital behavior keyboard 1012 may determine the composite cognitive measure as described in connection with sections V and VI. Determination of the composite cognitive measure is based on the user interaction features including the dynamic keystroke data. For instance, determining the composite cognitive measure may include calculating a mean of a sliding average of the autocorrect frequency and a median of the interkey delay measured over a time period.

At block 1108, the method 1100 includes displaying the composite cognitive measure on a portion of the digital behavior keyboard. For instance, the digital behavior keyboard 1012 may display the composite cognitive measure as an icon directly on a portion of the digital behavior keyboard concurrently with the keys. An example of such an icon is the icon 1062 (FIGS. 10A and 10B), which is displayed concurrently with the keys 1060.

In some examples, the method 1100 further includes generating an information page overlaid onto the digital behavior keyboard that displays details of the composite cognitive measure. For example, the digital behavior keyboard 1012 may display the icon 1063A on an information page overlaid onto the digital behavior keyboard 1012 as shown in FIG. 10C. The information page may include additional details as well, such as a daily timeline (e.g., by hour) of the composite cognitive measure.

In further examples, the method 1100 includes receiving a selection of the details of the composite cognitive measure, and based on receiving the selection of the details of the composite cognitive measure, increasing a size of the information page overlaid onto the digital behavior keyboard and displaying user data on the information page while the size of the information page is increased. For instance, the digital behavior keyboard 1012 may receive a selection of the icon 1063A, and display the timeline region 1065 and/or the event region 1066 in an increased size information page, as illustrated by FIG. 10D. As described in connection with section VI, these regions may include user data such as a timeline and/or events.

In some examples, the digital behavior keyboard includes a life events page. For example, the digital behavior keyboard 1012 includes the event editing region 1068 on a separate page of the digital behavior keyboard 1012. In such examples, the user data may include life events entered via the keys (e.g., the keys 1060).

Within examples, the method 1100 includes displaying a control for entry of group identifier codes via the application, receiving, via the displayed control, a group identifier code, associating the user with a particular group of users that are associated with the group identifier code, and displaying a comparison of the composite cognitive measure for the user with a representation of a composite cognitive measure for the particular group of users. The method 1100 may further include displaying a control for entry of user identifiers and receiving, via the displayed control, a user identifier that uniquely identifies the user within a group of other users.

In some examples, the method 1100 includes displaying a control that is selectable to generate a cognitive report. The cognitive report may include a graph or other graphical representation of the composite cognitive measure over a pre-determined period of time along with related timeline information. The cognitive report is electronically sharable (e.g., via the Internet) with medical professionals and/or other users. An example of such a control is the report icon 1071.

In some examples, the dynamic keystroke data is stored in a database of the computing device, and where the stored dynamic keystroke data excludes user-identifying information.

In some examples, the dynamic keystroke data is stored in a remote server, where the dynamic keystroke data excludes user-identifying information, where the remote server also stores additional dynamic keystroke data associated with a plurality of additional users.

In some examples, the computing device comprises a physical keyboard and/or a user display capable of receiving user input, where the keystroke dynamic data is collected using the physical keyboard and/or a keyboard displayed on the user display of the computing device.

In some examples, the computing device is a mobile computing device.

In some examples, the sensors comprise an accelerometer, a gyroscope, or both the accelerometer and the gyroscope, and where the user interaction features is partially or entirely collected from the accelerometer, the gyroscope, or both the accelerometer and the gyroscope.

### CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid state drive, or another storage medium.

The computer readable medium can also include non-transitory computer readable media such as computer readable media that store data for short periods of time like register memory and processor cache. The computer readable media can further include non-transitory computer readable media that store program code and/or data for longer periods of time. Thus, the computer readable media may include secondary or persistent long-term storage, like ROM, optical or magnetic disks, solid state drives, or compact-disc read only memory (CD-ROM), for example. The computer readable media can also be any other volatile or non-volatile storage systems. A computer readable medium can be considered a computer readable storage medium, for example, or a tangible storage device.

When any of the appended claims are read to cover a purely software and/or firmware implementation, at least one of the elements in at least one example is expressly defined to include a tangible, non-transitory medium such as a memory, DVD, CD, Blu-ray, or the like, storing the software and/or firmware.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the appended claims.

Example 1: A method comprising: initiating, on a computing device, an application that adds a digital behavior keyboard to the computing device, wherein, when enabled, the digital behavior keyboard replaces a digital application keyboard on the computing device, and wherein the digital behavior keyboard comprises keys that are selectable via a touchscreen of the computing device to provide input to other applications; while the digital behavior keyboard is enabled, measuring, via the digital behavior keyboard, user interaction features with the digital behavior keyboard, wherein the user interaction features comprise dynamic keystroke data representative of user keyboard usage patterns made while providing keystroke input via the keys to one or more of the other applications; based on the user interaction features including the dynamic keystroke data, determining, via the digital behavior keyboard, a composite cognitive measure for a user; and displaying, by the computing device on the touchscreen, the composite cognitive measure as an icon directly on a portion of the digital behavior keyboard concurrently with the keys.

Example 2: The method of Example 1, further comprising: based on receiving a selection of the icon, generating an information page overlaid onto the digital behavior keyboard that displays details of the composite cognitive measure.

Example 3: The method of Example 2, further comprising: receiving a selection of the details of the composite cognitive measure; and based on receiving the selection of the details of the composite cognitive measure, increasing a size of the information page overlaid onto the digital behavior keyboard and displaying user data on the information page while the size of the information page is increased.

Example 4: The method of Example 3, wherein the displayed user data comprises a graphical timeline of the composite cognitive measure.

Example 5: The method of Example 3, wherein the graphical timeline of the composite cognitive measure comprises a selectable icon showing the composite cognitive measure for a current time period, and wherein receiving the selection of the details of the composite cognitive measure comprises receiving an input representing a selection of the selectable icon.

Example 6: The method of Example 3, wherein the digital behavior keyboard further comprises a life event page, and wherein the user data includes life events entered via the keys.

Example 7: The method of Example 3, further comprising: receiving, via the touchscreen, input data representing at least one of (a) a scroll or (b) a swipe; and based on receiving the input data, modifying the information page to display additional information associated with the user.

Example 8: The method of Example 3, further comprising: displaying a control for entry of group identifier codes via the application; receiving, via the displayed control, a group identifier code; based on receiving the group identifier code, associating the user with a particular group of users that are associated with the group identifier code; and displaying a comparison of the composite cognitive measure for the user with a representation of a composite cognitive measure for the particular group of users.

Example 9: The method of Example 8, further comprising: displaying a control for entry of user identifiers; and receiving, via the displayed control, a user identifier that uniquely identifies the user within a group of other users.

Example 10: The method of Example 10, further comprising: displaying a control that is selectable to generate a cognitive report which includes a graph of the composite cognitive measure over a pre-determined period of time along with related timeline information, wherein the cognitive report is electronically sharable.

Example 11: The method of any of Examples 1-10, wherein the dynamic keystroke data includes interkey delay data.

Example 12: The method of Example 11, wherein the dynamic keystroke data includes autocorrect frequency, and wherein determining the composite cognitive measure comprises calculating a mean of a sliding average of the autocorrect frequency and a median of the interkey delay measured over a time period.

Example 13: The method of Example 11, further comprising: recording a time between successive keystrokes as the interkey delay data based on a first input corresponding to a first keystroke and a second input corresponding to a second keystroke, wherein the first keystroke and the second keystrokes corresponding to press of at least one key among the keys.

Example 14: The method of claim 1, wherein determining the composite cognitive measure comprises: applying a dual slope linear scaling which increases the slope of the composite cognitive measure when the composite cognitive measure is less than the midpoint and decreases the slope of the composite cognitive measure when the composite cognitive measure is greater than the midpoint.

Example 15: At least one non-transitory computer-readable storage medium having stored thereon program instructions that, upon execution by at least one processor, configure a computing device to perform the method of any of Examples 1-14.

Example 16: A computing device comprising a touchscreen; at least one processor; and at least one non-transitory computer-readable storage medium having stored thereon program instructions that, upon execution by the at least one processor, configure the computing device to perform the method of any of Examples 1-14.

Example 17: A computing system configured to perform the method of any of Examples 1-14.

Example 18: A server configured to facilitate the method of any of Examples 1-14.

What is claimed is:

1. A method comprising:
initiating, on a computing device, an application that adds a digital behavior keyboard to the computing device, wherein, when enabled, the digital behavior keyboard replaces a digital application keyboard on the computing device, and wherein the digital behavior keyboard comprises keys that are selectable via a touchscreen of the computing device to provide input to other applications;
while the digital behavior keyboard is enabled, measuring, via the digital behavior keyboard, user interaction features with the digital behavior keyboard, wherein the user interaction features comprise dynamic keystroke data representative of user keyboard usage patterns made while providing keystroke input via the keys to one or more of the other applications;
based on the user interaction features including the dynamic keystroke data, determining, via the digital behavior keyboard, a composite cognitive measure for a user; and
displaying, by the computing device on the touchscreen, the composite cognitive measure as an icon directly on a portion of the digital behavior keyboard concurrently with the keys.

2. The method of claim 1, further comprising:
based on receiving a selection of the icon, generating an information page overlaid onto the digital behavior keyboard that displays details of the composite cognitive measure.

3. The method of claim 2, further comprising:
receiving a selection of the details of the composite cognitive measure; and
based on receiving the selection of the details of the composite cognitive measure, increasing a size of the information page overlaid onto the digital behavior keyboard and displaying user data on the information page while the size of the information page is increased.

4. The method of claim 3, wherein the displayed user data comprises a graphical timeline of the composite cognitive measure.

5. The method of claim 3, wherein the graphical timeline of the composite cognitive measure comprises a selectable icon showing the composite cognitive measure for a current time period, and wherein receiving the selection of the details of the composite cognitive measure comprises receiving an input representing a selection of the selectable icon.

6. The method of claim 3, wherein the digital behavior keyboard further comprises a life event page, and wherein the user data includes life events entered via the keys.

7. The method of claim 3, further comprising:
receiving, via the touchscreen, input data representing at least one of (a) a scroll or (b) a swipe; and
based on receiving the input data, modifying the information page to display additional information associated with the user.

8. The method of claim 3, further comprising:
displaying a control for entry of group identifier codes via the application;
receiving, via the displayed control, a group identifier code;
based on receiving the group identifier code, associating the user with a particular group of users that are associated with the group identifier code; and
displaying a comparison of the composite cognitive measure for the user with a representation of a composite cognitive measure for the particular group of users.

9. The method of claim 8, further comprising:
displaying a control for entry of user identifiers; and
receiving, via the displayed control, a user identifier that uniquely identifies the user within a group of other users.

10. The method of claim 3, further comprising:
displaying a control that is selectable to generate a cognitive report which includes a graph of the composite cognitive measure over a pre-determined period of time along with related timeline information, wherein the cognitive report is electronically sharable.

11. The method of claim 1, wherein the dynamic keystroke data includes interkey delay data.

12. The method of claim 11, wherein the dynamic keystroke data includes autocorrect frequency, and wherein determining the composite cognitive measure comprises calculating a mean of a sliding average of the autocorrect frequency and a median of the interkey delay measured over a time period.

13. The method of claim 11, further comprising:
recording a time between successive keystrokes as the interkey delay data based on a first input corresponding to a first keystroke and a second input corresponding to a second keystroke, wherein the first keystroke and the second keystrokes corresponding to press of at least one key among the keys.

14. The method of claim 1, wherein determining the composite cognitive measure comprises:
applying a dual slope linear scaling which increases the slope of the composite cognitive measure when the composite cognitive measure is less than the midpoint and decreases the slope of the composite cognitive measure when the composite cognitive measure is greater than the midpoint.

15. A computing device comprising:
a touchscreen;
at least one processor; and
at least one non-transitory computer-readable storage medium having stored thereon program instructions that, upon execution by the at least one processor, configure the computing device to:
initiate an application that adds a digital behavior keyboard to the computing device, wherein, when enabled, the digital behavior keyboard replaces a digital application keyboard on the computing device, and wherein the digital behavior keyboard comprises keys that are selectable via the touchscreen of the computing device to provide input to other applications;
while the digital behavior keyboard is enabled, measure, via the digital behavior keyboard, user interaction features with the digital behavior keyboard, wherein the user interaction features comprise dynamic keystroke data representative of user keyboard usage patterns made while providing keystroke input via the keys to one or more of the other applications;
based on the user interaction features including the dynamic keystroke data, determine, via the digital behavior keyboard, a composite cognitive measure for a user; and
display, by the computing device on the touchscreen, the composite cognitive measure as an icon directly on a portion of the digital behavior keyboard concurrently with the keys.

16. At least one non-transitory computer-readable storage medium having stored thereon program instructions that, upon execution by at least one processor, configure a computing device to:
initiate an application that adds a digital behavior keyboard to the computing device, wherein, when enabled, the digital behavior keyboard replaces a digital application keyboard on the computing device, and wherein the digital behavior keyboard comprises keys that are selectable via a touchscreen of the computing device to provide input to other applications;
while the digital behavior keyboard is enabled, measure, via the digital behavior keyboard, user interaction features with the digital behavior keyboard, wherein the user interaction features comprise dynamic keystroke data representative of user keyboard usage patterns made while providing keystroke input via the keys to one or more of the other applications;
based on the user interaction features including the dynamic keystroke data, determine, via the digital behavior keyboard, a composite cognitive measure for a user; and
display, by the computing device on the touchscreen, the composite cognitive measure as an icon directly on a portion of the digital behavior keyboard concurrently with the keys.

17. The at least one non-transitory computer-readable storage medium of claim 16, wherein the at least one non-transitory computer-readable medium further comprises program instructions that that, upon execution by at least one processor, configure the computing device to:
based on receipt of a selection of the icon, generate an information page overlaid onto the digital behavior keyboard that displays details of the composite cognitive measure.

18. The at least one non-transitory computer-readable storage medium of claim 17, wherein the at least one non-transitory computer-readable medium further comprises program instructions that that, upon execution by at least one processor, configure the computing device to:
receive a selection of the details of the composite cognitive measure; and
based on receipt of the selection of the details of the composite cognitive measure, increase a size of the information page overlaid onto the digital behavior keyboard and displaying user data on the information page while the size of the information page is increased.

19. The at least one non-transitory computer-readable storage medium of claim 18, wherein the displayed user data comprises a graphical timeline of the composite cognitive measure.

20. The at least one non-transitory computer-readable storage medium of claim 18, wherein the graphical timeline of the composite cognitive measure comprises a selectable icon showing the composite cognitive measure for a current time period, and wherein receiving the selection of the details of the composite cognitive measure comprises receiving an input representing a selection of the selectable icon.
